(19) United States
(12) Patent Application Publication
(10) Pub. No.: US 2019/0358345 A1
(43) Pub. Date: Nov. 28, 2019

(54) AMPHIPHILIC NONSTEROID ANTI-INFLAMMATORY PLATINUM NANOPARTICLES AND PREPARATION METHODS THEREFOR

(71) Applicant: CHINA PHARMACEUTICAL UNIVERSITY, Nanjing, Jiangsu (CN)

(72) Inventors: Hulin JIANG, Nanjing, Jiangsu (CN); Lei XING, Nanjing, Jiangsu (CN); Chenxi YANG, Nanjing, Jiangsu (CN)

(73) Assignee: CHINA PHARMACEUTICAL UNIVERSITY, Nanjing, Jiangsu (CN)

(21) Appl. No.: 16/324,911

(22) PCT Filed: Sep. 4, 2017
(86) PCT No.: PCT/CN2017/100356

§ 371 Date: Feb. 12, 2019

(30) Foreign Application Priority Data
Jul. 27, 2017 (CN) 201710623558.9

(51) Int. Cl. A61K 47/69; A61K 47/55; A61K 31/555; A61K 31/635

(52) U.S. Cl. CPC A61K 47/6929; A61K 31/635; A61K 31/555; A61K 47/55

(57) ABSTRACT

The present invention discloses amphiphilic nonsteroid anti-inflammatory platinum nanoparticles, preparation methods therefor and applications thereof. The amphiphilic nonsteroid anti-inflammatory platinum nanoparticle includes an amphiphilic complex formed by coordinating a nonsteroid anti-inflammatory drug and a platinum antitumor drug. Compared with other platinum drugs, the amphiphilic nonsteroid anti-inflammatory platinum prepared according to the present invention can self-assemble into a nanoparticle in an aqueous solution without the addition of an additive or a surfactant. The amphiphilic platinum complex can achieve targeted accumulation of tumor tissues by means of the enhanced permeability and retention effect of the tumor tissues, thereby reducing the side effects of platinum drugs. After entering cells under the phagocytosis of the tumor cells, the nonsteroid anti-inflammatory platinum nanoparticle can release two drugs under the hydrolysis effect, on the one hand, the nonsteroid anti-inflammatory drug can inhibit COX-2 and relieve the symptoms of inflammation, and on the other hand, the platinum drug enters the cells to inhibit the tumor cell proliferation, and thus the two drugs can collaboratively achieve the purpose of treating tumors.

### FIELD

The present invention relates to the technical field of biological medicines, and in particular, to amphiphilic nonsteroid anti-inflammatory platinum nanoparticles for treating tumors, preparation methods therefor and applications thereof.

### BACKGROUND

Malignant tumor is one of the leading causes of human death, and its treatment is still one of the greatest challenges in the world. At present, clinical treatment of tumors still mainly relies on surgical intervention, radiotherapy, chemotherapy, and immunotherapy. Chemotherapy is still one of the most common methods for clinical tumor treatment. antitumor platinum drugs are one of the most common chemotherapy drugs in clinical practice, especially cisplatin, oxaliplatin and carboplatin are first-line chemotherapy drugs, which was widely used in the treatment of malignant tumors (ovarian cancer, testicular cancer, lung cancer, bladder cancer, lymphoma, myeloma, melanoma, colorectal cancer, head-neck carcinoma, prostate cancer and the like). However, the currently clinical used antitumor platinum drugs have the following problems: poor stability, toxic and side effects (neurological toxicity, neurotoxicity, myelosuppression and the like) to normal tissues, rapid blood clearance, and drug resistance, etc. The foregoing problems promote a variety of novel and improved antitumor platinum drugs, and the use of nanotechnology for the targeted and synergistic delivery of platinum drugs has achieved the goal of enhancing antitumor and reducing the side effects. For example, lobaplatin which is marketed in China, eptaplatin sold in Korea, and nedaplatin which is licensed in Japan maintains a good therapeutic effect of platinum, and also reduces the corresponding toxicity. In addition, nano-delivery carriers such as liposomes, micelle, vesicles, polymers and inorganic materials change the distribution of antitumor platinum drugs in vivo, and achieve targeted delivery of platinum drugs in tumor tissues, in order to increase the concentration of drugs in the lesions, reduce the accumulation in normal tissues, and reduce side effects. However, some carrier materials may cause unnecessary side effects after the platinum drugs are delivered to tumor cells based on these nanocarriers, and the toxic effects of platinum itself are not obvious. In addition, the nano-delivery system has poor reproducibility and cannot be used for scale production. Therefore, the construction of a high-efficiency low-toxicity platinum drug delivery system is one of the important directions in the current research of platinum drugs.

At present, researches show that inflammation plays a role in promoting the occurrence, development and metastasis of tumors. Cyclooxygenase (COX) is a key rate-limiting enzyme during the production of inflammatory factors. After the cells are stimulated, cytomembranes are destroyed, the phospholipids are oxidized to arachidonic acid by means of phospholipase, and the COX can catalyze the conversion of arachidonic acid to prostaglandin, thromboxane and prostacyclin so as to induce the occurrence of inflammatory reaction. In recent years, COX-2 has become a new research target for tumor prevention and treatment. Clinical researches revealed that long-term use of Nonsteroid Anti-Inflammatory Drugs (NSAID), that is, COX inhibitors can reduce the incidence of colon cancer by 40-60%. COX-2 plays an important role in tumorigenesis, and is mainly related to the following points: 1. COX-2 is overexpressed in ovarian cancer, gastric cancer, colon cancer, lung cancer, breast cancer and other tumor tissues, and the expression intensity is correlated with tumor tissue type and degree of differentiation; 2, COX-2 in tumor cells is generally expressed synchronously with VEGF; and VEGF expression is up-regulated if the COX-2 expression is up-regulated, thereby promoting the formation and metastasis of malignant tumors; 3. the overexpression of the COX-2 can also up-regulate the expression of the anti-apoptotic factor BCL-2 and inhibit tumor cell apoptosis; and 4. The overexpression of the COX-2 in the tumor cells results in decreased sensitivity of cancer cells to drugs and radiation. Many of the foregoing evidences indicate that the COX-2 is highly correlated with tumors. Moreover, it has been reported that the COX-2 inhibitors have synergistical antitumor effects by inhibiting tumor cell cycle progression, inducing tumor cell apoptosis, and inhibiting tumor angiogenesis, etc. Traditional single-agent anticancer drugs are used in combination to achieve tumor treatment, but there are many drawbacks in this administration mode, e.g., it is difficult to simultaneously deliver two drugs to effective targets; the two drugs have independent parameters such as pharmacokinetics parameters and biodistribution and are inconvenient for the determination of drug content and efficacy evaluation in viva. Therefore, a new strategy for combined chemotherapy has emerged, i.e., constructing two drug complex as a prodrugs with antitumor effects, which are delivered to the drug targets in vivo and then dissociated from the original drug to exert their respective effects.

Therefore, the present invention aims at increasing the cellular uptake of antitumor platinum drugs, down-regulating the expression of the anti-apoptotic factor BCL-2, and shortening the repair time of damaged DNA to achieve synergistic anti-cancer effects. It has been reported that the COX-2 inhibitors reduce the self-repair time of DNA-damaged cells while effectively reducing the BCL-2 expression. Many COX-2 inhibitors have coordinating groups, which can coordinate with the antitumor platinum drugs to form an amphiphilic nonsteroid anti-inflammatory platinum complex, without affecting the coordination valence of platinum, and can also self-assemble into form nanoparticles in an aqueous solution, and therefore, the nanoparticles can increase the drug uptake through the high permeability and retention effect of tumor tissues, and are endocytosed into the tumor cells to release two kinds of drugs, thereby exerting the synergistic antitumor effect.

### SUMMARY

### Objective

to overcome the deficiencies in the prior art, the present invention provides an amphiphilic nonsteroid anti-inflammatory platinum nanoparticle to solve the technical problems such as the toxic effect brought by a carrier in a current platinum antitumor drug delivery system, the toxicity of a platinum drug itself, and poor reproducibility of nanoparticles.

### The Technical Solution

to solve the foregoing technical problems, the present invention adopts the following technical solutions:

an amphiphilic nonsteroid anti-inflammatory platinum nanoparticle, including a nonsteroid anti-inflammatory drug and an antitumor platinum drug, where the nonsteroid anti-inflammatory drug and the antitumor platinum drug are coordinated with each other and self-assemble into a nanoparticle in an aqueous solution, and the amphiphilic nonsteroid anti-inflammatory platinum nanoparticle is endocytosed into a tumor cell, and then is hydrolyzed to release the nonsteroid anti-inflammatory drug and the antitumor platinum drug.

As a preferred solution, the means particle size of the amphiphilic nonsteroid anti-inflammatory platinum nanoparticle is less than 200 nm, and more preferably, the means particle size of the amphiphilic nonsteroid anti-inflammatory platinum nanoparticle is 157 nm.

As a preferred solution, the nonsteroid anti-inflammatory drug is one or more of meloxicam, meclofenamic acid, mefenamic acid, niflumic acid, flunixin, clonixin, meloxicam, piroxicam, diclofenac, lomoxicam, tenoxicam, nimesulide, niflumic acid, ampiroxicam, flufenamic acid, amfenac, bromfenac, aceclofenac, ibuprofen, indomethacin, sulindac, o-acetylsalicylic acid, suprofen, flurbiprofen, etodolac, ketoprofen, naproxen, fenbufen, ketorolac, zaltoprofen, oxaprozin, acemetacin, etoricoxib, carprofen, pranoprofen, loxoprofen, sasapyrine, evodiamine, mesalazine, diflunisal, sulfasalazine, gentisic acid, olsalazine, fendosal, balsalazide, salicylic acid, meclofenamic acid, lumiracoxib, and tolfenamic acid.

As a preferred solution, the platinum antitumor drug is one or more of cisplatin, cyclohexane diamine dinitro platinum, cis-dichloro-1,2-cyclohexanediamine platinum, cis-dichloro-1,4-cyclohexanediamine platinum, cis-diiododiammine platinum, picoplatin, cis-dichloro-trans-ammonia(cyclohexylamine)platinum, cis-dichloro(cyclopropylamine)platinum, cis-dichloro(cyclopentylamine)platinum, cis-diiododiammine platinum, and chloroplatinic acid.

The present invention further provides a preparation method for the amphiphilic nonsteroid anti-inflammatory platinum nanoparticle, including the following steps:

(1) coordinating a nonsteroid anti-inflammatory drug with an antitumor platinum drug to obtain an amphiphilic nonsteroid anti-inflammatory platinum; and

(2) dissolving the amphiphilic nonsteroid anti-inflammatory platinum in an organic solvent, dropping into a stirred aqueous solution, and removing the organic solvent by dialysis to obtain an amphiphilic nonsteroid anti-inflammatory platinum nanoparticle solution.

Specifically, the coordination reaction in step (1) is selected from one of the following two methods:

method I: dispersing an antitumor platinum drug, a nonsteroid anti-inflammatory drug, silver nitrate, and alkali in an organic solvent, stirring and reacting in a dark place, centrifugally removing AgCl precipitate to obtain a supernatant, concentrating the supernatant, adding the organic solvent, and then washing with weak alkaline water and water, collecting an organic solvent layer, concentrating, and performing column purification to obtain an amphiphilic nonsteroid anti-inflammatory platinum; and

method II: dispersing an antitumor platinum drug, a nonsteroid anti-inflammatory drug, silver nitrate, and alkali in water, stirring and reacting in a dark place, centrifugally removing AgCl precipitate to obtain a supernatant, lyophilizing the supernatant, and performing column purification to obtain an amphiphilic nonsteroid anti-inflammatory platinum.

As a preferred solution, the preparation method for the amphiphilic nonsteroid anti-inflammatory platinum nanoparticle, where the molar ratio of the platinum antitumor drug, the nonsteroid anti-inflammatory drug to silver nitrate in the coordination reaction is 1:(0.5-1.05):(0-2.05), and the silver nitrate is not available in the actual coordination reaction.

As a preferred solution, the organic solvent is one or more of N,N′-dimethylformamide, dimethyl sulfoxide, N-methylpyrrolidone, ethanol, methanol, acetonitrile, acetone, and tetrahydrofuran; and the alkali is one or more of sodium carbonate, sodium bicarbonate, sodium hydroxide, potassium hydroxide, and triethylamine.

As a preferred solution, the aqueous solution is one or more of water, 5% glucose, and a phosphate buffer solution.

The present invention further provides an application of the amphiphilic nonsteroid anti-inflammatory platinum nanoparticle in preparation of a drug for treating tumors or drug-resistant tumors. Amphiphilic nonsteroid anti-inflammatory platinum nanoparticles can be administered orally, by pulmonary inhalation or by injection.

### Advantageous Effects

the amphiphilic nonsteroid anti-inflammatory platinum nanoparticles provided by the present invention can self-assemble into a nanoparticle in an aqueous solution. Without any excipient or additive, the nonsteroid anti-inflammatory platinum can achieve drug delivery by itself, and by means of the enhanced permeability and retention effect of the tumor tissues, the complex nanoparticles can enter the tumor tissues and the tumor cells more effectively, and reduce the side effects on the normal tissue cells. After the complex nanoparticles enter the tumor cells, the platinum drug and the nonsteroid anti-inflammatory drug are released by hydrolysis to enhance the anti-tumor effect. The high permeability and retention effect of the tumor tissues increases the drug intake, and after endocytosis into the tumor cells, two kinds of drugs are released, thereby exerting a synergistic anti-tumor effect.

### BRIEF DESCRIPTION OF THE DRAWINGS

FIG. 1 is a ¹H NMR spectrum of amphiphilic nonsteroid anti-inflammatory platinum drug synthesized in Embodiment 1 and Embodiment 2;

FIG. 2 is an ultraviolet-visible spectrum of amphiphilic nonsteroid anti-inflammatory platinum drug synthesized in Embodiment 1 and Embodiment 2;

FIG. 3 is a dynamic light scattering diagram of the amphiphilic nonsteroid anti-inflammatory platinum nanoparticles prepared in Embodiment 1 and Embodiment 2;

FIG. 4 is a graph showing a result of inhibiting the growth of MCF-7 tumor cells by the amphiphilic nonsteroid anti-inflammatory platinum nanoparticles prepared in Embodiment 1;

FIG. 5 is a result of inhibiting the growth of 4T1 tumor cells by the amphiphilic nonsteroid anti-inflammatory platinum nanoparticles prepared in Embodiment 1;

FIG. 6 is a result of inhibiting the growth of A549 tumor cells by the amphiphilic nonsteroid anti-inflammatory platinum nanoparticles prepared in Embodiment 1; and

FIG. 7 is a result of inhibiting the growth of A549/DDP tumor cells by the amphiphilic nonsteroid anti-inflammatory platinum nanoparticles prepared in Embodiment 1.

### DETAILED DESCRIPTION

The present invention will be further described below in conjunction with the accompanying drawings and specific embodiments. The specific embodiments are all implemented on the premise of the technical solutions of the present invention, including detailed description and specific operation procedures, but the scope of protection of the present invention is not limited to the following embodiments.

### Embodiment 1

Cisplatin (150 mg), AgNO₃ (170 mg), tolfenamic acid (131 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and then the solution is washed three times with 50 ml of 0.1 M Na₂CO₃ and washed three times with water, and an ethyl acetate layer is collected and dried with anhydrous sodium sulfate overnight. After filtration, the filtrate is separated by silica gel column chromatography using an eluent of methanol:chloroform=1:9 to obtain a cisplatin-tofinic acid complex in a yield of 60%, and the structural formula is represented by Formula 1.

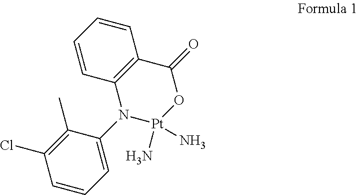

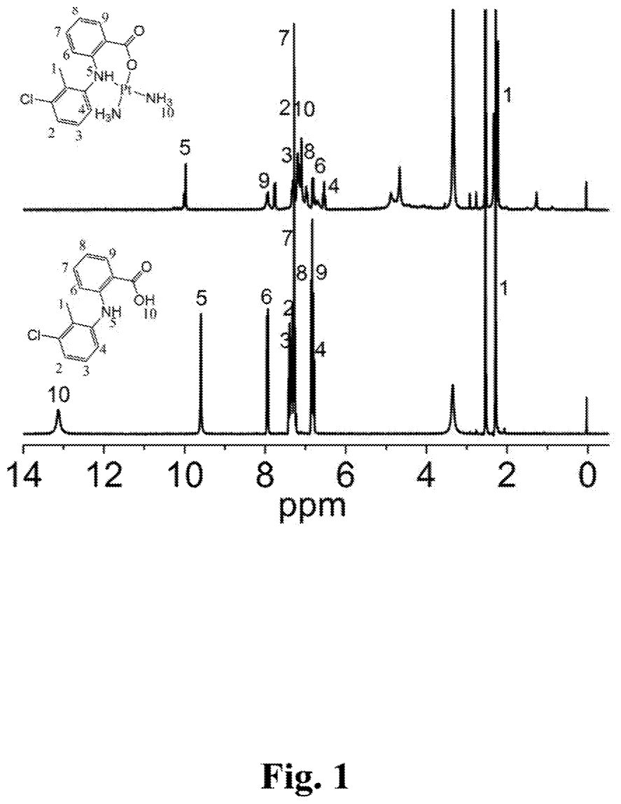

The chemical structure of the complex synthesized in this embodiment is as shown in Formula 1. The ¹H NMR spectrum of the complex prepared in this embodiment is shown in FIG. 1. The test solvent is d6-DMSO, and the respective absorption peaks are assigned.

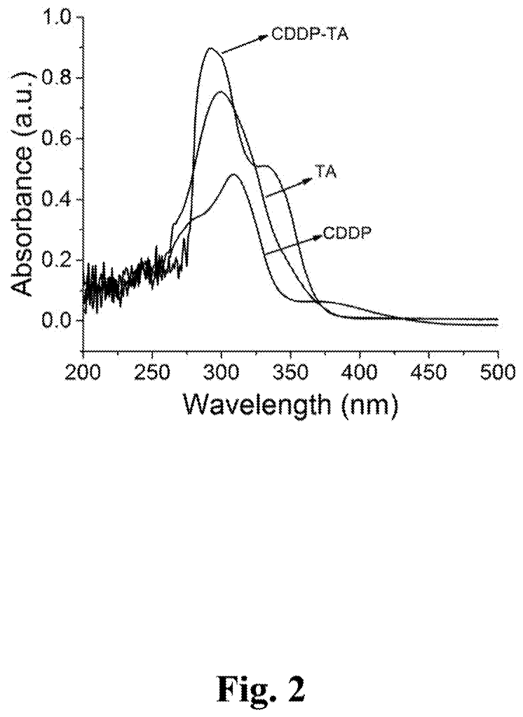

The foregoing synthesized amphiphilic nonsteroid anti-inflammatory platinum is dissolved in DMF, and subjected to ultraviolet-visible scanning in the range of 200-450 nm on a ultraviolet-visible spectrophotometer. The ultraviolet-visible spectrum of the cisplatin-tolfenamic acid-based complex prepared in this embodiment is shown in FIG. 2. The maximum ultraviolet-visible absorption wavelength of cisplatin-tolfenamic acid is 292 nm, and there is a shoulder peak at about 340 nm.

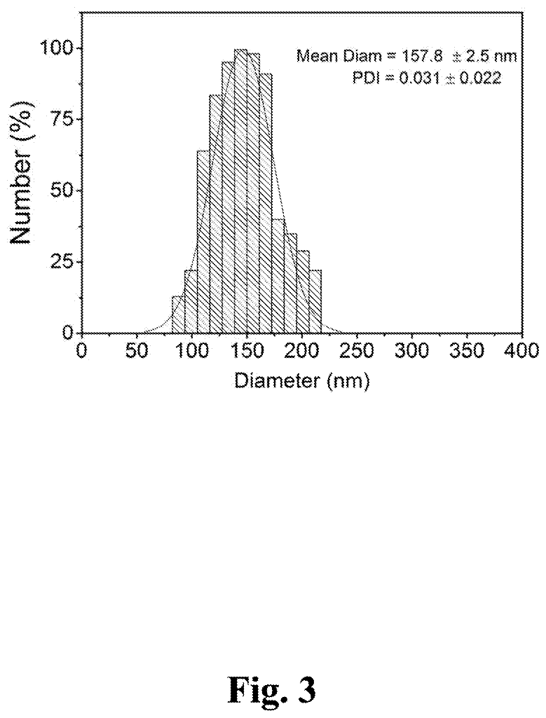

The foregoing amphiphilic nonsteroid anti-inflammatory platinum is dissolved in DMF, dropped into an aqueous solution at room temperature, and the DMF is removed by dialysis to obtain an amphiphilic nonsteroid anti-inflammatory platinum nanoparticle solution. The dynamic light scattering results of the antitumor cisplatin-tonifamic acid nanoparticles prepared in this embodiment are shown in FIG. 3, and the means particle size of the nanoparticles is 157 nm.

### Embodiment 2

CDDP (150 mg), AgNO₃ (113 mg), tolfenamic acid (131 mg) and Na₂CO₃ (35.3 mg) are added to a round-bottom flask, then 10 ml of water is added and stirred at room temperature for 24 h in a dark place. The AgCl precipitate is centrifugally discarded, and the filtrate is dried in vacuum. The crude product is purified by silica gel column chromatography using an eluent of methanol:chloroform=1:9 to obtain the cisplatin-tolfenamic acid-based complex in a yield of 53%. The chemical structure of the complex synthesized in this embodiment is as shown in Formula 1. The ¹H NMR spectrum of the complex prepared in this embodiment is shown in FIG. 1, and the ultraviolet-visible spectrum of the cisplatin-tolfenamic acid-based complex prepared in this embodiment is shown in FIG. 2, and the dynamic light scattering result of the antitumor cisplatin-tolfenamic acid nanoparticles prepared in this embodiment is shown in FIG. 3, and the means particle size of the nanoparticles is 157 nm.

### Embodiment 3

Cyclohexane diamine dinitro platinum (216.6 mg), lumiracoxib (146.9 mg) and NaHCO₃ (42 mg) are added to a round-bottom flask, and then 15 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a lumiracoxib-cyclohexanediamine dinitroplatinum complex in a yield of 63%, and the structural formula is represented by Formula 2.

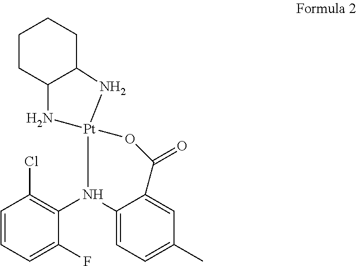

### Embodiment 4

Cis-dichloro-1,2-cyclohexanediamine platinum (190 mg), AgNO₃ (170 mg), niflumic acid (141 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:ethyl acetate=0.8:9.2 to obtain a niflumic acid-cyclohexanediamine platinum complex in a yield of 56%, and the structural formula is represented by Formula 3.

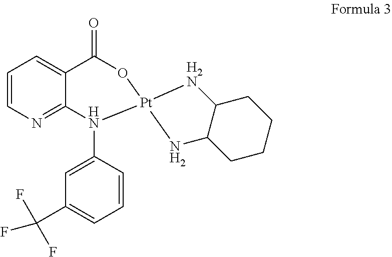

### Embodiment 5

Cis-dichloro-1,4-cyclohexanediamine platinum (190 mg), AgNO₃ (170 mg), niflumic acid (141 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a niflumic acid-cyclohexanediamine platinum complex in a yield of 56%, and the structural formula is represented by Formula 4.

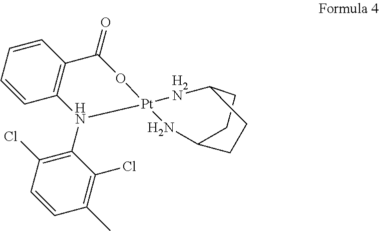

### Embodiment 6

Cis-diiododiammine platinum (241.5 mg), AgNO₃ (170 mg), salicylic acid (69 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a salicylic acid-cisplatin complex in a yield of 56%, and the structural formula is represented by Formula 5.

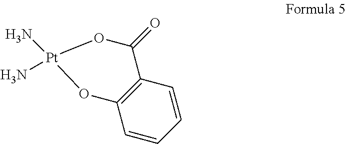

### Embodiment 7

Picoplatin (188 mg), AgNO₃ (170 mg), diflunisal (125 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a diflunisal-picoplatin complex in a yield of 53%, and the structural formula is represented by Formula 6.

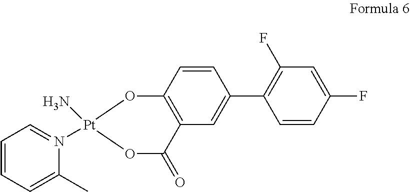

### Embodiment 8

Chloroplatinic acid (204.9 mg), AgNO₃ (170 mg), evodiamine (143.7 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain an evodiamine-platinum complex in a yield of 73%, and the structural formula is represented by Formula 7.

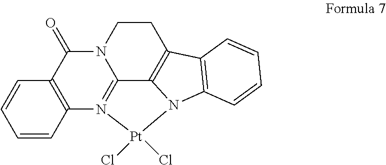

### Embodiment 9

Cis-dichloro-trans-ammonia(cyclohexylamine)platinum (191 mg), AgNO₃ (170 mg), sulfasalazine (199.2 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a sulfasalazine-ammonia(cyclohexylamine)platinum complex in a yield of 55%, and the structural formula is represented by Formula 8.

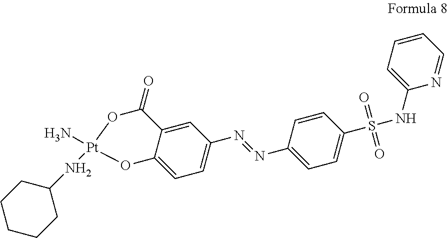

### Embodiment 10

Cis-dichloro(cyclopropylamine)platinum (161.5 mg), AgNO₃ (170 mg), flufenamic acid (140.6 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a flufenamic acid-cyclopropylamine platinum complex in a yield of 58%, and the structural formula is represented by Formula 9.

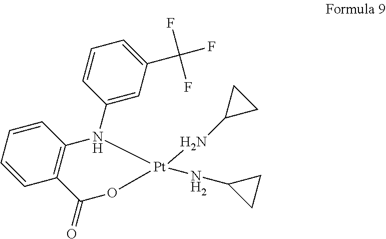

### Embodiment 11

Cis-dichloro(cyclopentylamine)platinum (175.6 mg), AgNO₃ (170 mg), meclofenamic acid (148 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a meclofenamic acid-cyclopentylamine platinum complex in a yield of 58%, and the structural formula is represented by Formula 10.

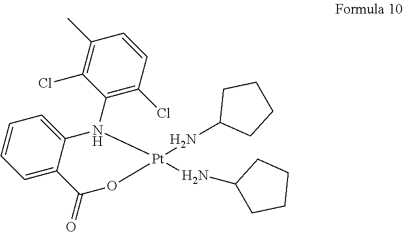

### Embodiment 12

Cisplatin (150 mg), AgNO₃ (170 mg), mefenamic acid (120.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₁ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-mefenamic acid complex in a yield of 67%, and the structural formula is represented by Formula 11.

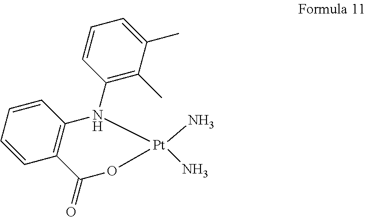

### Embodiment 13

Cisplatin (150 mg), AgNO₃ (170 mg), niflumic acid (141 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-niflumic acid complex in a yield of 68%, and the structural formula is represented by Formula 12.

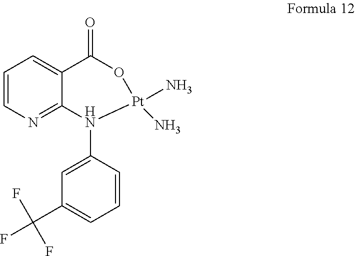

### Embodiment 14

Cisplatin (150 mg), AgNO₃ (170 mg), flunixin (148 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-flunixin complex in a yield of 60%, and the structural formula is represented by Formula 13.

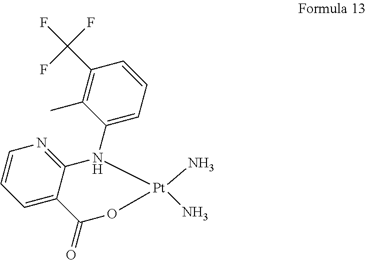

### Embodiment 15

Cisplatin (150 mg), AgNO₃ (170 mg), clonixin (131.4 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-clonixin complex in a yield of 62%, and the structural formula is represented by Formula 14.

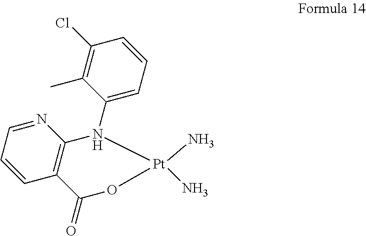

### Embodiment 16

Cisplatin (150 mg), AgNO₃ (170 mg), meloxicam (175.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform—1:9 to obtain a cisplatin-meloxicam complex in a yield of 60%, and the structural formula is represented by Formula 15.

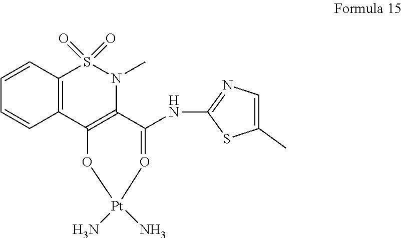

### Embodiment 17

Cisplatin (150 mg), AgNO₃ (170 mg), piroxicam (165.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-piroxicam complex in a yield of 58%, and the structural formula is represented by Formula 16.

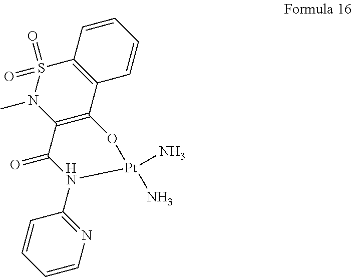

### Embodiment 18

Cis-dichloro-1,2-cyclohexanediamine platinum (190 mg), AgNO₃ (43 mg), diclofenac (148 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a diclofenac-cyclohexanediamine platinum complex in a yield of 50%, and the structural formula is represented by Formula 17.

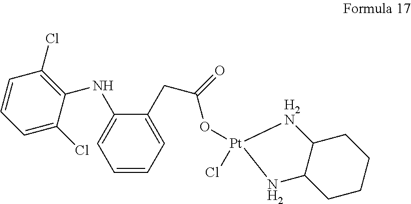

### Embodiment 19

Cisplatin (150 mg), AgNO₃ (170 mg), lomoxicam (185.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-lornoxicam complex in a yield of 63%, and the structural formula is represented by Formula 18.

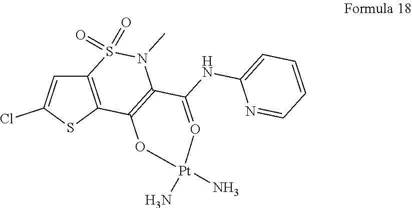

### Embodiment 20

Cisplatin (150 mg), AgNO₃ (170 mg), tenoxicam (168.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-tenoxicam complex in a yield of 67%, and the structural formula is represented by Formula 19.

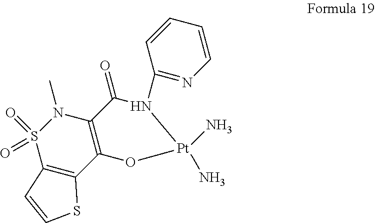

### Embodiment 21

Cisplatin (150 mg), AgNO₃ (170 mg), nimesulide (168.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-nimesulide complex in a yield of 67%, and the structural formula is represented by Formula 20.

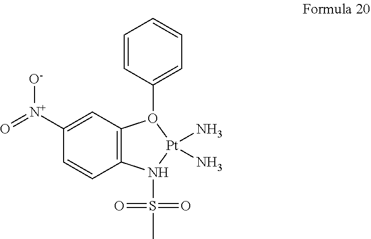

### Embodiment 22

Cisplatin (150 mg), AgNO₃ (170 mg), niflumic acid (141 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-niflumic acid complex in a yield of 69%, and the structural formula is represented by Formula 21.

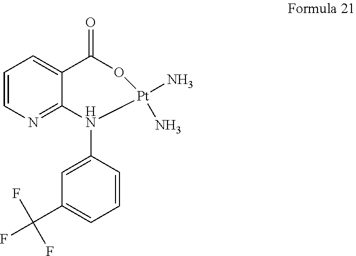

### Embodiment 23

Cisplatin (150 mg), AgNO₃ (170 mg), ampiroxicam (223.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-ampiroxicam complex in a yield of 65%, and the structural formula is represented by Formula 22.

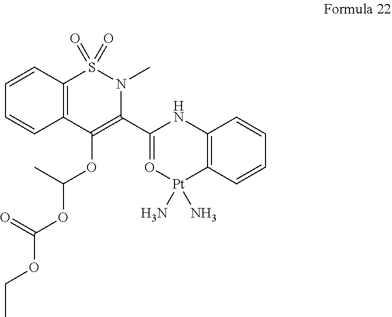

### Embodiment 24

Cisplatin (150 mg), AgNO₃ (170 mg), flufenamic acid (140.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-flufenamic acid complex in a yield of 68%, and the structural formula is represented by Formula 23.

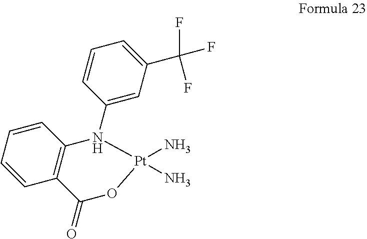

### Embodiment 25

Cisplatin (150 mg), AgNO₃ (170 mg), gentisic acid (77 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a cisplatin-gentisic acid complex in a yield of 53%, and the structural formula is represented by Formula 24.

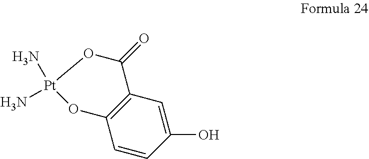

### Embodiment 26

Cisplatin (150 mg), AgNO₃ (170 mg), olsalazine (75.6 mg) and Na₂CO₃ (70.6 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a cisplatin-olsalazine complex in a yield of 49%, and the structural formula is represented by Formula 25.

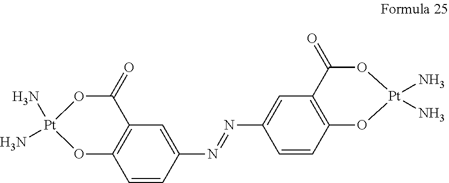

### Embodiment 27

Cisplatin (150 mg), AgNO₃ (170 mg), fendosal (190.7 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a cisplatin-fendosal complex in a yield of 53%, and the structural formula is represented by Formula 26.

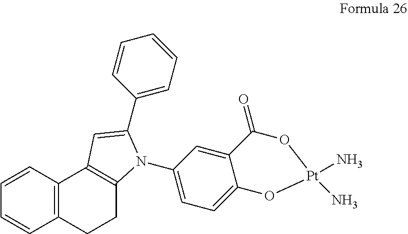

### Embodiment 28

Cisplatin (150 mg), AgNO₃ (170 mg), balsalazide (178.7 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a cisplatin-balsalazide complex in a yield of 52%, and the structural formula is represented by Formula 27.

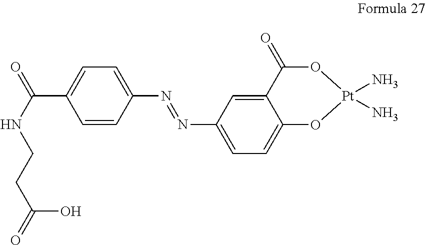

### Embodiment 29

Cis-dichloro-1,2-cyclohexanediamine platinum (190 mg), AgNO₃ (43 mg), amfenac (136.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and then the afterwards, is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a amfenac-cyclohexanediamine platinum complex in a yield of 49%, and the structural formula is represented by Formula 28.

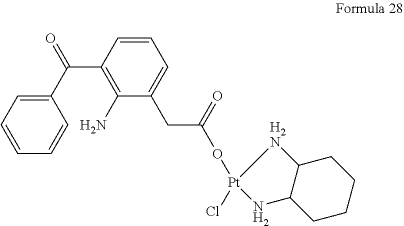

### Embodiment 30

Cisplatin (150 mg), AgNO₃ (170 mg), bromfenac (167 mg) and Na₂CO₃ (35.3 mg) are added to a round-bottom flask, and then 15 ml of DMF is added and stirred at room temperature for 24 h in a dark place. The precipitate is centrifugally discarded, and the filtrate is concentrated, and then 50 ml of ethyl acetate is added, and afterwards, the solution is washed three times with 50 ml of 0.1 M Na₂CO₃, and washed three times with water, and the ethyl acetate layer is collected and dried with anhydrous sodium sulfate overnight. After filtration, the filtrate is separated by silica gel column chromatography using an eluent of methanol:dichloromethane=1:9 to obtain a cisplatin-bromfenac complex in a yield of 50%, and the structural formula is represented by Formula 29.

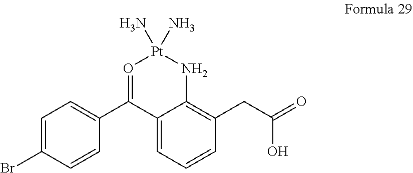

### Embodiment 31

Cis-dichloro-1,2-cyclohexanediamine platinum (190 mg), AgNO₃ (43 mg), aceclofenac (177 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an aceclofenac-cyclohexanediamine platinum complex in a yield of 50%, and the structural formula is represented by Formula 30.

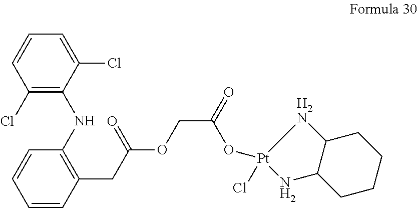

### Embodiment 32

Cis-dichloro-1,2-cyclohexanediamine platinum (190 mg), AgNO₃ (43 mg), ibuprofen (103 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an ibuprofen-cyclohexanediamine platinum complex in a yield of 50%, and the structural formula is represented by Formula 31.

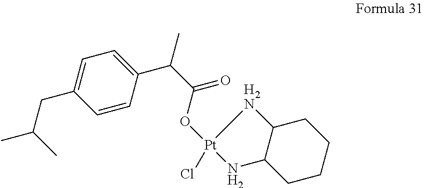

### Embodiment 33

Cis-dichloro-1,2-cyclohexanediamine platinum (190 mg), AgNO₃ (43 mg), indomethacin (178.9 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an indomethacin-cyclohexanediamine platinum complex in a yield of 50%, and the structural formula is represented by Formula 32.

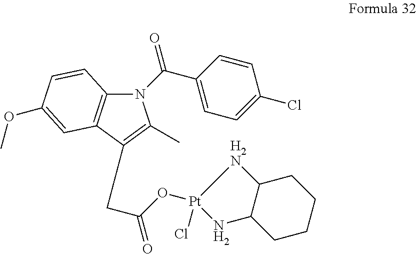

### Embodiment 34

Cis-dichloro-1,2-cyclohexanediamine platinum (190 mg), AgNO₃ (43 mg), sulindac (178.2 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a sulindac-cyclohexanediamine platinum complex in a yield of 50%, and the structural formula is represented by Formula 33.

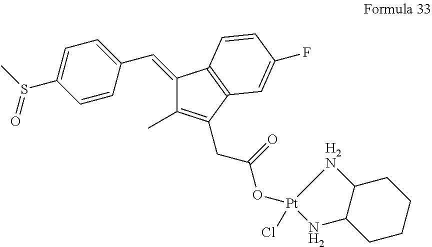

### Embodiment 35

Cis-dichloro-1,2-cyclohexanediamine platinum (190 mg), AgNO₃ (43 mg), o-acetylsalicylic acid (90 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an o-acetylsalicylic acid-cyclohexanediamine platinum complex in a yield of 56%, and the structural formula is represented by Formula 34.

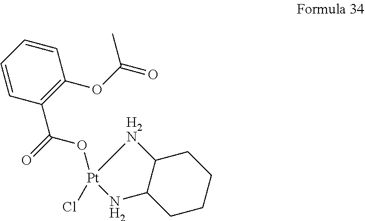

### Embodiment 36

Cis-dichloro-1,2-cyclohexanediamine platinum (190 mg), AgNO₃ (43 mg), suprofen (130 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a suprofen-cyclohexanediamine platinum complex in a yield of 55%, and the structural formula is represented by Formula 35.

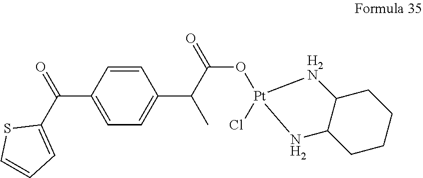

### Embodiment 37

Cis-dichloro-1,2-cyclohexanediamine platinum (190 mg), AgNO₃ (43 mg), flurbiprofen (122 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a flurbiprofen-cyclohexanediamine platinum complex in a yield of 56%, and the structural formula is represented by Formula 36.

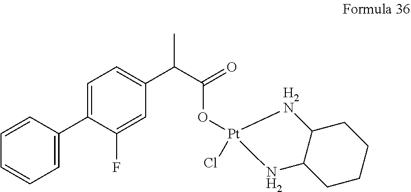

### Embodiment 38

Cis-dichloro-1,2-cyclohexanediamine platinum (190 mg), AgNO₃ (43 mg), etodolac (143.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an etodolac-cyclohexanediamine platinum complex in a yield of 52%, and the structural formula is represented by Formula 37.

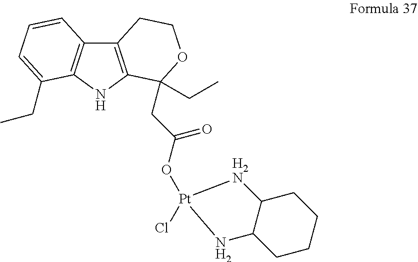

### Embodiment 39

Cis-dichloro-1,2-cyclohexanediamine platinum (190 mg), AgNO₃ (43 mg), ketoprofen (127 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a ketoprofen-cyclohexanediamine platinum complex in a yield of 52%, and the structural formula is represented by Formula 38.

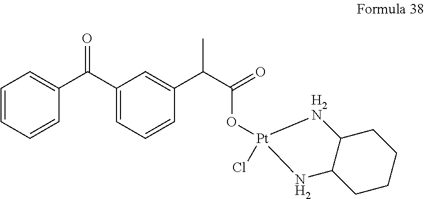

### Embodiment 40

Cis-dichloro-1,2-cyclohexanediamine platinum (190 mg), AgNO₃ (43 mg), naproxen (115 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a naproxen-cyclohexanediamine platinum complex in a yield of 56%, and the structural formula is represented by Formula 39.

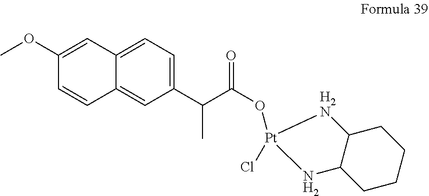

### Embodiment 41

Cis-dichloro-1,2-cyclohexanediamine platinum (190 mg), AgNO₃ (43 mg), fenbufen (127 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a fenbufen-cyclobexanediamine platinum complex in a yield of 56%, and the structural formula is represented by Formula 40.

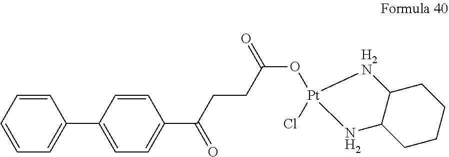

### Embodiment 42

Cis-dichloro-1,2-cyclohexanediamine platinum (190 mg), AgNO₃ (43 mg), ketorolac (127.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a ketorolac-cyclohexanediamine platinum complex in a yield of 55%, and the structural formula is represented by Formula 41.

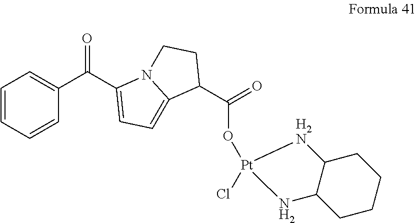

### Embodiment 43

Cis-dichloro-1,2-cyclohexanediamine platinum (190 mg), AgNO₃ (43 mg), zaltoprofen (149 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a zaltoprofen-cyclohexanediamine platinum complex in a yield of 52%, and the structural formula is represented by Formula 42.

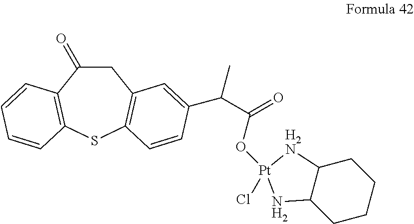

### Embodiment 44

Cis-dichloro-1,2-cyclohexanediamine platinum (190 mg), AgNO₃ (43 mg), oxaprozin (146.7 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an oxaprozin-cyclohexanediamine platinum complex in a yield of 55%, and the structural formula is represented by Formula 43.

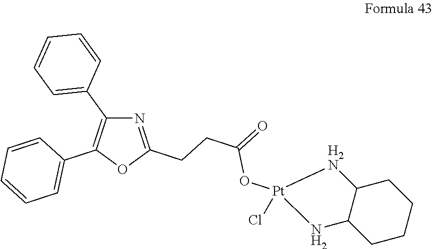

### Embodiment 45

Cis-dichloro-1,2-cyclohexanediamine platinum (190 mg), AgNO₃ (43 mg), acemetacin (207.9 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain an acemetacin-cyclohexanediamine platinum complex in a yield of 53%, and the structural formula is represented by Formula 44.

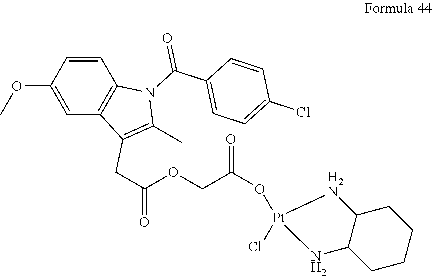

### Embodiment 46

Cisplatin (300 mg), AgNO₃ (86 mg), and etoricoxib (177.9 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of saturated sodium chloride and washed three times with water, and the ethyl acetate layer is collected and dried with anhydrous sodium sulfate overnight. After filtration, the filtrate is separated by silica gel column chromatography using an eluent of methanol:chloroform=1:9 to obtain an etoricoxib-cisplatin complex in a yield of 60%, and the structural formula is represented by Formula 45.

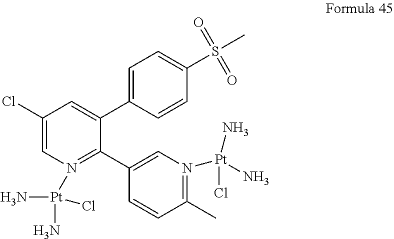

### Embodiment 47

Cis-dichloro-1,2-cyclohexanediamine platinum (190 mg), AgNO₃ (43 mg), carprofen (136.8 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a carprofen-cyclohexanediamine platinum complex in a yield of 56%, and the structural formula is represented by Formula 46.

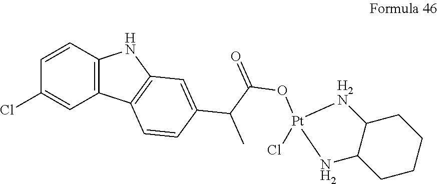

### Embodiment 48

Cis-dichloro-1,2-cyclohexanediamine platinum (190 mg), AgNO₃ (43 mg), pranoprofen (127.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a pranoprofen-cyclohexanediamine platinum complex in a yield of 52%, and the structural formula is represented by Formula 47.

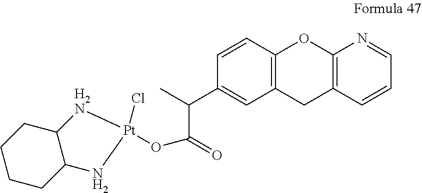

### Embodiment 49

Cis-dichloro-1,2-cyclohexanediamine platinum (190 mg), AgNO₃ (43 mg), loxoprofen (123 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a loxoprofen-cyclohexanediamine platinum complex in a yield of 53%, and the structural formula is represented by Formula 48.

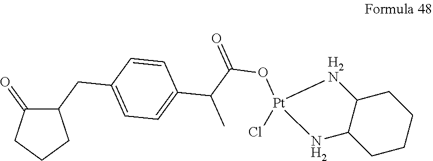

### Embodiment 50

Cis-dichloro-1,2-cyclohexanediamine platinum (190 mg), AgNO₃ (43 mg), sasapyrine (129 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a bisalicylate-cyclohexanediamine platinum complex in a yield of 52%, and the structural formula is represented by Formula 49.

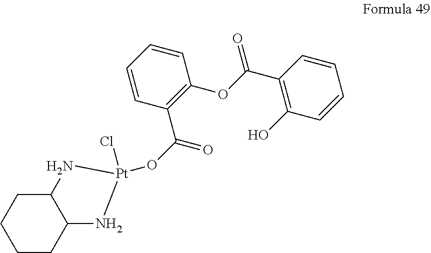

### Embodiment 51

Cisplatin (150 mg), AgNO₃ (170 mg), mesalazine (76.6 mg) and Na₂CO₃ (35.3 mg) are added to a round-bottom flask, then 10 ml of DMF is added and stirred at room temperature for 24 h in a dark place. The AgCl precipitate is centrifugally discarded, and the filtrate is concentrated, and then 50 ml of ethyl acetate is added, and afterwards, the solution is washed three times with 50 ml of 0.1 M Na₂CO₃ and washed three times with water, and the ethyl acetate layer is collected and dried with anhydrous sodium sulfate overnight. After filtration, the filtrate is separated by silica gel column chromatography using an eluent of methanol:chloroform=1:9 to obtain a cisplatin-mesalazine complex in a yield of 60%, and the structural formula is represented by Formula 50.

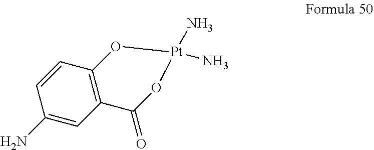

Experiments on effects of the amphiphilic nonsteroid anti-inflammatory platinum nanoparticle for treating tumors of the present invention on cancer cells:

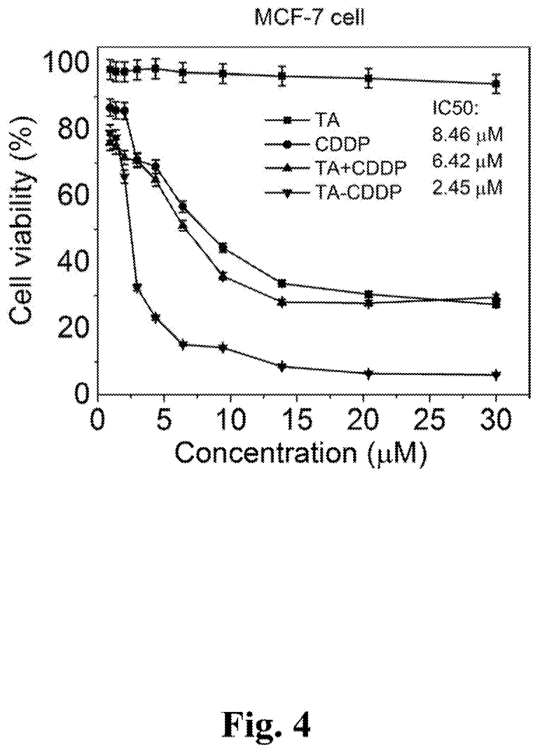

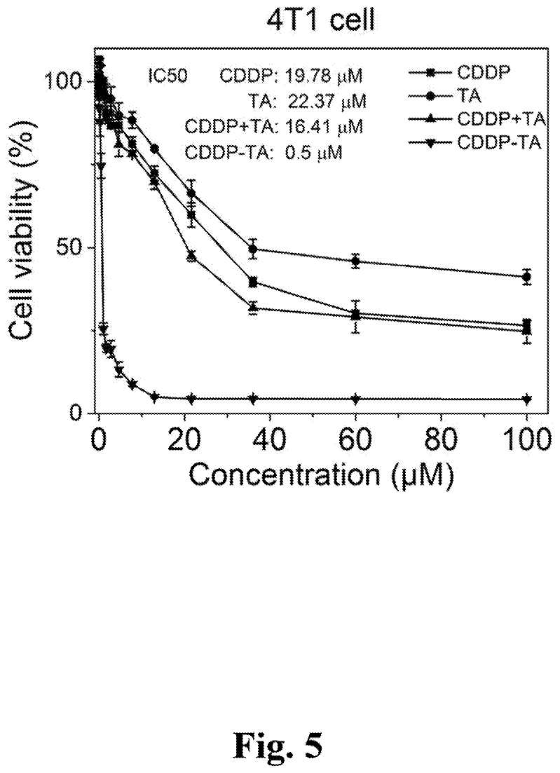

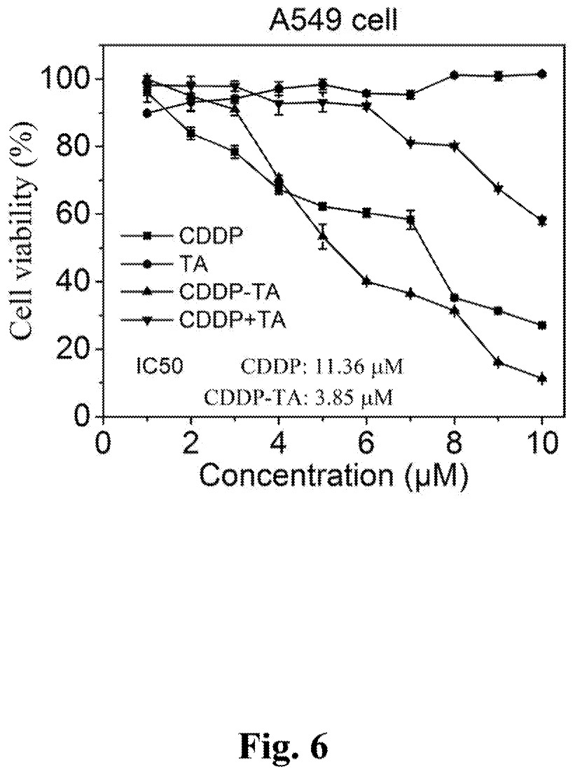

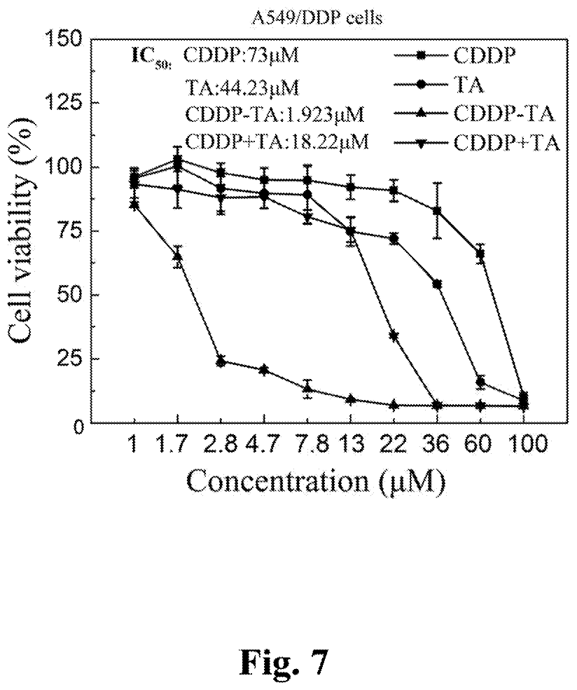

the amphiphilic nonsteroid anti-inflammatory platinum nanoparticles prepared in Embodiment 1 are separately diluted with a cell culture medium into solutions of different concentrations, and then are separately cultured in MCF-7 (human breast cancer cells), 4T1 (murine breast cancer cells), A549 (human non-small cell lung cancer cells) and A549/DDP (cisplatin-resistant human non-small cell lung cancer cells) for 24 h, and cell viabilities are tested using an MTT method. The results are shown in FIGS. 4, 5, 6, and 7. When the IC₅₀ of the amphiphilic nonsteroid anti-inflammatory platinum nanoparticles are 2.45 μM, 0.5 μM, 3.85 μM and 1.923 μM, respectively, these results show that the cisplatin-tofenamic acid-based complex nanoparticles have a strong ability of killing cancer cells. It is indicated that the amphiphilic nonsteroid anti-inflammatory platinum nanoparticles have great potential and application prospects in the treatment of malignant tumors and even drug-resistant tumors.

The descriptions above are merely preferred embodiments of the present invention, and it should be noted that those skilled in the art can make several improvements and modifications, including salt formation, structural modification, and isomers of the crude drugs, without departing from the principles of the present invention, and such improvements and modifications should also be considered as the protection scope of the present invention.

1. An amphiphilic nonsteroid anti-inflammatory platinum nanoparticle, comprising a nonsteroid anti-inflammatory drug and a platinum antitumor drug, wherein the nonsteroid anti-inflammatory drug and the platinum antitumor drug are coordinated with each other and self-assembled into a nanoparticle in an aqueous solution, and the amphiphilic nonsteroid anti-inflammatory platinum nanoparticle is endocytosed into a tumor cell, and then is hydrolyzed to release the nonsteroid anti-inflammatory drug and the platinum antitumor drug.

2. The amphiphilic nonsteroid anti-inflammatory platinum nanoparticle according to claim 1, wherein,
the means particle size of the amphiphilic nonsteroid anti-inflammatory platinum nanoparticle is less than 200 nm, and/or the means particle size of the amphiphilic nonsteroid anti-inflammatory platinum nanoparticle is 157 nm.

3. The amphiphilic nonsteroid anti-inflammatory platinum nanoparticle according to claim 1, wherein
the nonsteroid anti-inflammatory drug is one or more of meloxicam, meclofenamic acid, mefenamic acid, niflumic acid, flunixin, clonixin, meloxicam, piroxicam, diclofenac, lornoxicam, tenoxicam, nimesulide, niflumic acid, ampiroxicam, flufenamic acid, amfenac, bromfenac, aceclofenac, ibuprofen, indomethacin, sulindac, o-acetylsalicylic acid, suprofen, flurbiprofen, etodolac, ketoprofen, naproxen, fenbufen, ketorolac, zaltoprofen, oxaprozin, acemetacin, etoricoxib, carprofen, pranoprofen, loxoprofen, sasapyrine, evodiamine, mesalazine, diflunisal, sulfasalazine, gentisic acid, olsalazine, fendosal, balsalazide, salicylic acid, meclofenamic acid, lumiracoxib, and tolfenamic acid.

4. The amphiphilic nonsteroid anti-inflammatory platinum nanoparticle according to claim 1, wherein
the antitumor platinum drug is one or more of cisplatin, cyclohexane diamine dinitro platinum, cis-dichloro-1,2-cyclohexanediamine platinum, cis-dichloro-1,4-cyclohexanediamine platinum, cis-diiododiammine platinum, picoplatin, cis-dichloro-trans-ammonia(cyclohexylamine)platinum, cis-dichloro(cyclopropylamine)platinum, cis-dichloro(cyclopentylamine)platinum, cis-diiododiammine platinum, and chloroplatinic acid.

5. A preparation method for the amphiphilic nonsteroid anti-inflammatory platinum nanoparticle according to claim 1, comprising the following steps:
(1) coordinating a nonsteroid anti-inflammatory drug with an antitumor platinum drug to obtain an amphiphilic nonsteroid anti-inflammatory platinum; and
(2) dissolving the amphiphilic nonsteroid anti-inflammatory platinum in an organic solvent, dropping into a stirred aqueous solution, and removing the organic solvent by dialysis to obtain an amphiphilic nonsteroid anti-inflammatory platinum nanoparticle solution.

6. The preparation method for the amphiphilic nonsteroid anti-inflammatory platinum nanoparticle according to claim 5, wherein the coordination reaction in step (1) is selected from one of the following two methods:
method I: dispersing an antitumor platinum drug, a nonsteroid anti-inflammatory drug, silver nitrate, and alkali in an organic solvent, stirring and reacting in a dark place, centrifugally removing AgCl precipitate to obtain a supernatant, concentrating the supernatant, adding the organic solvent, and then washing with weak alkaline water and water, collecting an organic solvent layer, concentrating, and performing column purification to obtain an amphiphilic nonsteroid anti-inflammatory platinum; and
method II: dispersing an antitumor platinum drug, a nonsteroid anti-inflammatory drug, silver nitrate, and alkali in water, stirring and reacting in a dark place, centrifugally removing AgCl precipitate to obtain a supernatant, lyophilizing the supernatant, and performing column purification to obtain an amphiphilic nonsteroid anti-inflammatory platinum.

7. The preparation method for the amphiphilic nonsteroid anti-inflammatory platinum nanoparticle according to claim 6, wherein
the molar ratio of the antitumor platinum drug, the nonsteroid anti-inflammatory drug to the silver nitrate in the coordination reaction is 1:(0.5-1.05):(0-2.05).

8. The preparation method for the amphiphilic nonsteroid anti-inflammatory platinum nanoparticle according to claim 6, wherein
the organic solvent is one or more of N,N′-dimethylformamide, dimethyl sulfoxide, N-methylpyrrolidone, ethanol, methanol, acetonitrile, acetone, and tetrahydrofuran; and the alkali is one or more of sodium carbonate, sodium bicarbonate, sodium hydroxide, potassium hydroxide, and triethylamine.

9. The preparation method for the amphiphilic nonsteroid anti-inflammatory platinum nanoparticle according to claim 5, wherein
the aqueous solution is one or more of water, 5% glucose, and a phosphate buffer solution.

10. An application of the amphiphilic nonsteroid anti-inflammatory platinum nanoparticle according to claim 1 in preparation of a drug for treating tumors or drug-resistant tumors.
